(19) United States
(12) Patent Application Publication
(10) Pub. No.: US 2020/0222952 A1
(43) Pub. Date: Jul. 16, 2020

(54) METHOD FOR RAPIDLY REDUCING ANTIBIOTICS AND ANTIBIOTIC RESISTANCE GENES IN ORGANIC SOLID WASTE

(71) Applicant: FUJIAN AGRICULTURE AND FORESTRY UNIVERSITY, Fuzhou (CN)

(72) Inventors: Shungui ZHOU, Fuzhou (CN); Hanpeng LIAO, Fuzhou (CN); Zhi CHEN, Fuzhou (CN)

(21) Appl. No.: 16/826,250

(22) Filed: Mar. 22, 2020

Related U.S. Application Data
(63) Continuation of application No. PCT/CN2018/073424, filed on Jan. 19, 2018.

(30) Foreign Application Priority Data
Nov. 27, 2017 (CN) 201711205616.2

(51) Int. Cl. B09B 3/00; C12N 1/20

(52) U.S. Cl. CPC B09B 3/00; C12N 1/20

(57) ABSTRACT

The present disclosure discloses a method for rapidly reducing antibiotics and antibiotic resistance genes in organic solid waste, which belongs to the field of organic solid waste treatment. The present disclosure utilizes aerobic fermentation bacteria which can tolerate a temperature of at least 80° C., and controls the compost pile temperature of no lower than 80° C. to perform hyperthermophilic composting on the organic solid waste for at least 5 to 7 days, so as to obtain effects of rapidly and stably reducing antibiotics and antibiotic resistance genes in organic solid waste.

### CROSS REFERENCE OF RELATED APPLICATIONS

This application is a continuation of PCT Patent Application No. PCT/CN2018/073424, filed on Jan. 19, 2018, entitled “METHOD FOR RAPIDLY REDUCING ANTIBIOTICS AND ANTIBIOTIC RESISTANCE GENES IN ORGANIC SOLID WASTE,” which claims foreign priority of Chinese Patent Application No. 201711205616.2, filed Nov. 27, 2017, in the China National Intellectual Property Administration, the entire contents of which are incorporated herein in their entirety.

### TECHNICAL FIELD

The present disclosure relates to the field of organic solid waste treatment, and particularly relates to a method for rapidly reducing antibiotics and antibiotic resistance genes in solid waste.

### BACKGROUND

China is the largest producer and consumer of antibiotics in the world. In 2013, the total amount of antibiotics used in China reached 162,000 tons, half of which were used in livestock and poultry breeding. In order to prevent disease and infection of livestock and poultry, farmers often feed large doses of antibiotics, and most of these antibiotics are not absorbed. About 30-90% of them are excreted in the form of active compound or metabolites with feces and urine, causing antibiotic residues in livestock and poultry feces exceeding standards. The unreasonable use and long-term abuse of antibiotics caused the antibiotic residues in the environment to exceed the standard, resulting in increased selection pressure of resistant microorganisms in the environment, leading to the contents of antibiotic resistance bacteria (ARB) and antibiotic resistance genes (ARGs) in the environment increasing. And antibiotic resistance genes can be spread between parents and other strains through inheritance and horizontal gene transfer, causing harm to human health and ecological balance. Since Pruden et al. proposed ARGs as a new type of environmental pollutants in 2006, there have been increasing reports about the pollution and spread of ARGs in the environment. The World Health Organization has identified ARGs as one of the major challenges to human health in the 21st century. Studies have shown that antibiotic resistance genes are detected in different ecological environments, such as the application of urban sludge soil, urban rivers, paddy soil applied pig manure, and even urban tap water.

Sewage sludge is the product of wastewater treatment plant. It is an extremely complex heterogeneous body composed of organic debris, bacterial cells, inorganic particles, colloids, and so on. The main characteristics of sludge are high water content (up to 99%), high organic content, easy to rot and odor, and a colloidal liquid. It is a thick substance between liquid and solid that can be transported by a pump, but it is difficult to separate solid and liquid. At present, the annual sludge production in China is about 35 million tons, and it is increasing at an annual rate of 10%-15%. Dewatered sludge often contains relatively high antibiotic residues and abundance of resistance genes, which has brought increasing environmental pollution problems. Current research shows that the existing various sewage treatment plant processes have a certain effect of removing antibiotic resistance genes in sewage. For example, Chinese patent No. 201510116821.6 discloses a coagulation method for removing antibiotic resistance genes in sewage sludge. It is mainly to remove ARGs in sewage water by coagulation, but the removal of ARGs in sewage sludge obtained by sedimentation and separation from the sewage water is not mentioned. In fact, in the prior art, the treatment process of the wastewater treatment plant is only responsible for the treatment of water, and there is no supporting for the harmless treatment process of the remaining sludge. Since sewage sludge and wastewater are two kinds of pollutants with completely different properties, the existing technology for removing ARG in sewage water cannot be applied to the treatment of ARGs in sewage sludge.

In addition, the form of sludge is between liquid and solid, which is somewhat similar to soil. However, because sludge is difficult to separate from solid and liquid, and the content material in sludge are completely different from that in soil, the ARGs removal technology in related soils cannot be used for ARGs in sludge. Because of the special properties of sludge, sludge is a kind of solid waste that is more difficult to handle than sewage water and soil. Therefore, it is necessary to study the prevention and control of sludge resistance gene pollution with a focused target and put forward a suitable resistance control technology combined with the existing resource treatment process of sludge.

In addition to sludge, organic solid waste includes a variety of poultry manure and fecal sewage, and antibiotic resistance genes have been detected. In particular, the abundance of ARGs in livestock and poultry manure is particularly high, reaching 1×10⁸ copies/g, so it is of great significance to develop antibiotic resistance gene reduction technology suitable for the above organic solid waste.

Thermophilic aerobic composting is the main approach for the utilization of organic solid wastes. The traditional thermophilic composting technology generally maintains the fermentation temperature at 55-65° C., which is mainly used to remove manure harmful materials, like weed seeds, Escherichia coli pathogenic bacteria in sludge. In recent years, a large number of studies have shown that traditional thermophilic compositing cannot effectively remove the resistance genes, but instead increases the content of some ARGs. ARGs are carried on the DNA or plasmids of host bacteria. The way to reduce ARGs is to directly degrade their DNA sequences, and more importantly to prevent them from spreading to other non-host microorganisms through gene transfer. Gene transfer of ARGs through mobile genetic elements is the main reason for the spread of ARGs everywhere. Xie et al. reported that after long-term application of organic fertilizer made from traditional sludge compost, the content of ARGs in farmland soil increased significantly, and there was a risk of expanding pollution and spread. So far, there is still a lack of efficient and economical treatment technology, which can not only solve the pollution of ARGs and antibiotic in organic solid waste, but also use waste material as resources.

Therefore, there is an urgent demand to develop an efficient and economical technical means: at the same time address the potential health risks caused by the pollution and spread of ARGs and antibiotics in organic solid waste, and also treat organic solid waste as a resource.

### SUMMARY

The object of the present disclosure is to provide an application of new technique hyperthermophilic composting for reducing antibiotics and antibiotic resistance genes in organic solid waste.

Another object of the present disclosure is to provide a method for rapidly reducing antibiotics and antibiotic resistance genes in organic solid waste;

Another object of the present disclosure is to provide a hyperthermophilic composting microbial inoculant and application thereof.

The technical solution adopted by the present disclosure is as follows:

The application of hyperthermophilic composting for reducing antibiotics and antibiotic resistance genes in organic solid waste, wherein the hyperthermophilic composting is to add an appropriate amount of hyperthermophilic composting microbial inoculant composed of aerobic microorganisms that are able to tolerate at least 80° C. into the organic solid waste to conduct the hyperthermophilic composting, and the temperature of the material is controlled to be not less than 80° C. for fermentation at least 5 to 7 days.

In some embodiments, the aerobic microorganisms that are able to tolerate at least 80° C. comprises at least one of Calditerricola yamamurae, Thermus thermophilus, Geobacillus sp., Bacillus methylotrophicus, and Bacillus sp.

In some embodiments, the hyperthermophilic composting microbial inoculant composed of aerobic microorganisms that are able to tolerate at least 80° C. is one or more selected from the group consisting of Calditerricola yamamurae UTM801 deposited as CGMCC No. 6185, Thermus thermophilus FAFU013 deposited as CGMCC No. 14654, Geobacillus sp. UTM801 deposited as CGMCC No. 5641, Bacillus methylotrophicus UTM401 deposited as CGMCC No. 5927, and Bacillus sp. UTM03 deposited as CGMCC No. 5643. The strains have been deposited and disclosed in related patents.

In some embodiments, the hyperthermophilic composting microbial inoculant that are able to tolerate at least 80° C. is obtained from by mixing Calditerricola yamamurae UTM801, Thermus thermophilus FAFU013, Geobacillus sp. UTM801, Bacillus methylotrophicus UTM401, and Bacillus sp. UTM03 according to the masse ratio of 1:1:1:1:1.

In some embodiments, the organic solid waste includes sludge, livestock and poultry manure, kitchen waste, etc.

In some embodiments, the antibiotic includes tetracycline, sulfadiazine, and oxytetracycline; and the resistance gene includes tetA, tetG, strA, strB, aacA4, aadE, ermT, mefA, and ereA.

Thermophilic composting (belonging to the category of thermophilic aerobic fermentation, which is the specific application of aerobic fermentation) refers to the process of degradation or transformation of organic matter in organic solid waste into stable humus by relying on the metabolism of obligate aerobic microorganism and facultative aerobic microorganism, mixing organic solid waste and auxiliary material in a certain proportion, And the microorganisms reproduce and degrade the organic matter under appropriate water and ventilation conditions, resulting in high temperature to kill pathogens and weed seeds, so that the organic matter to achieve stability.

The existing conventional aerobic composting technology is mainly used to remove the weed seeds, Escherichia coli and other pathogens of the organic solid waste, so as to achieve the stabilization and harmless indicators. However, it is not related to the elimination of antibiotics and resistance genes. Moreover, the highest temperature of the compost is less than 65° C., which may increase the content of heat resistant ARGs.

Through research, it is found that by using hyperthermophilic bacteria inoculant that can tolerate at least 80° C. and controlling the temperature of the compost pile to ferment at 80° C. or above for at least 5 to 7 days, the effect of rapidly reducing antibiotics and antibiotic resistance genes in organic solid waste can be obtained. The hyperthermophilic composting can not only rapidly reduce antibiotics and ARGs, but also change the whole microbial community structure in the compost pile, kill 90% of the microorganisms carrying ARGs, reduce the diversity of non-host microorganisms, thereby reducing the risk of gene transfer of ARGs, the spread of ARGs is controlled from the source, so as to ensure that ARGs will not rebound. However, the traditional high-temperature compost cannot control the spread of ARGs from the source. In addition, the main technical conditions of composting include regulation of organic matter content, moisture content, appropriate ventilation system, adjustment of C/N ratio and C/P ratio of auxiliary material, appropriate pH value, etc., which can be adjusted according to the existing technology and common knowledge.

The inventor Zhou, Shungui et al. carried out the research on isolation and screening of extremely thermophilic microorganisms and their functions, collected samples from a variety of extremely high temperature environments, and isolated more than 50 thermophilic bacterial strains. Through the substrate utilization and strain compounding test, the main functions and interrelations of most of the extreme thermophilic strains were basically defined, and several strains with strong organic material degradation ability were screened out. The aerobic fermentation bacteria used in the embodiment of the present disclosure is extremely thermophilic microorganism. The above thermophilic microorganism does not rely on external heat source for heating, but only uses the biological heat energy released by its metabolism and decomposition of organic solid waste to generate extreme high temperature (no less than 80° C.). Such high temperature can not only degrade ARGs directly, but also change the microbial community structure in the process of composting, reduce the abundance of bacteria (including host and non-host carrying ARGs), so as to prevent the path of large-scale spread of ARGs.

A method for rapidly reducing antibiotics and antibiotic resistance genes in organic solid waste, including adding an appropriate amount of hyperthermophilic composting microbial inoculant composed of aerobic microorganisms that are able to tolerate at least 80° C. into organic solid waste to conduct the hyperthermophilic composting, and controlling the temperature of the material to be not less than 80° C. for fermentation at least 5 to 7 days.

In some embodiments, the aerobic microorganisms that are able to tolerate at least 80° C. includes at least one of Calditerricola yamamurae, Thermus thermophilus, Geobacillus sp., Bacillus methylotrophicus, and Bacillus sp.

In some embodiments, the hyperthermophilic composting microbial inoculant composed of aerobic microorganisms that are able to tolerate at least 80° C. is one or more selected from the group consisting of Calditerricola yamamurae UTM801 deposited as CGMCC No. 6185, Thermus thermophilus FAFU013 deposited as CGMCC No. 14654, Geobacillus sp. UTM801 deposited as CGMCC No. 5641, Bacillus methylotrophicus UTM401 deposited as CGMCC No. 5927, and Bacillus sp. UTM03 deposited as CGMCC No. 5643.

In some embodiments, the hyperthermophilic composting microbial inoculant that are able to tolerate at least 80° C. is obtained by mixing Calditerricola yamamurae UTM801, Thermus thermophilus FAFU013, Geobacillus sp. UTM801, Bacillus methylotrophicus UTM401, and Bacillus sp. UTM03 according to the masse ratio of 1:1:1:1:1.

In some embodiments, the organic solid waste includes sludge, livestock and poultry manure, crop straw, etc.

In some embodiments, the antibiotic includes tetracycline, sulfadiazine, and oxytetracycline; and the resistance gene includes tetA, tetG, strA, strB, aacA4, aadE, ermT, mefA, and ereA.

In some embodiments, when conducting the hyperthermophilic composting, regulating the aeration rate and controlling the number of compost turning.

In some embodiments, wherein regulating the aeration rate and controlling the number of compost turning includes:

(1) controlling the initial aeration rate to 20 to 40 m³·t⁻¹·h⁻¹;

(2) regulating the aeration rate to 45-75% of the initial aeration rate after the material reaches the maximum temperature of 80° C. or above, keeping the temperature at 80° C. or above 80° C. for 5 to 7 days and no compost turning is performed during this process;

(3) regulating the aeration rate to 35-50% of the initial amount after the hyperthermophilic composting above 80° C. is completed, and turning compost pile once in 5-7 days; when the temperature drops to room temperature, the fermentation is ended.

In some embodiments, before the organic solid waste is added with an appropriate amount of hyperthermophilic composting inoculant for hyperthermophilic composting, the water content is required regulation, and the water content of the organic solid waste shall be regulated to 50% to 65% by adding an appropriate amount of back mixing material or auxiliary material.

In some embodiments, the auxiliary material include rice husk, straw, fermented and decomposed materials, etc.

In some embodiments, there is no need to adjust the C/N ratio of the organic solid waste before the hyperthermophilic composting.

In some embodiments, the added amount of hyperthermophilic composting inoculant is 0.05 to 0.1% of the mass of the mixed materials.

In some embodiments, the initial aeration rate is 20 to 30 m³·t⁻¹·h⁻¹.

In some embodiments, regulating the aeration rate to 50 to 70% of the initial aeration rate when the material temperature reaches 80° C. or above.

In some embodiments, regulating the aeration rate to 35 to 45% of the initial aeration rate after the hyperthermophilic composting at 80° C. or above is completed, and turning compost pile once a week;

The method of the present disclosure is preferably used for treating more than 100 tons of organic solid waste in one batch.

An hyperthermophilic composting microbial inoculant, calculated by mass percentage, wherein the active ingredients of the hyperthermophilic composting microbial inoculant includes Calditerricola yamamurae, Thermus thermophilus FAFU013, Geobacillus sp., Bacillus methylotrophicus, and Bacillus sp.

In some embodiments, the hyperthermophilic composting microbial inoculant includes at least one of Calditerricola yamamurae, Thermus thermophilus, Geobacillus sp., Bacillus methylotrophicus, and Bacillus sp.

In some embodiments, the hyperthermophilic composting microbial inoculant is one or more selected from the group consisting of Calditerricola yamamurae UTM801 CGMCC No. 6185, Thermus thermophilus FAFU013 deposited as CGMCC No. 14654, Geobacillus sp. UTM801 deposited as CGMCC No. 5641, Bacillus methylotrophicus UTM401 deposited as CGMCC No. 5927, and Bacillus sp. UTM03 deposited as CGMCC No. 5643.

In some embodiments, the hyperthermophilic composting microbial inoculant is obtained by mixing Calditerricola yamamurae UTM801, Thermus thermophilus FAFU013, Geobacillus sp. UTM801, Bacillus methylotrophicus UTM401, and Bacillus sp. UTM03 according to the masse ratio of 1:1:1:1:1.

The application of the hyperthermophilic composting microbial inoculant according to any solution mentioned above for reducing antibiotics and antibiotic resistance genes in organic solid waste.

The advantageous effects of the present disclosure are shown as below.

The present disclosure utilizes aerobic fermentation bacteria that can tolerate at least 80° C., and controls the temperature of the compost pile to be fermented at not less than 80° C. for at least 5 to 7 days to perform hyperthermophilic composting on organic solid waste, which can quickly and stably reduce the antibiotics and resistance genes in organic solid waste. The hyperthermophilic composting can not only rapidly degrade antibiotics and ARGs, but also change the entire microbial community structure in the compost, kill 90% of the microorganisms carrying ARGs, reduce the risk of gene transfer of ARGs, and control its spread from the source. so as to guaranteed that ARGs will not rebound. It was found through analysis that the removal of ARGs was mainly due to the change in the structure of the microbial community, which reduced the spread risk, especially reducing the diversity index of host bacteria carrying ARGs in the compost pile.

The method of the present disclosure does not require external heating, and only relies on the self metabolism of the thermophilic microorganism to supply energy to reach the high temperature of fermentation, which has advantages in low energy consumption and environment friendly.

The effect of the present disclosure for processing antibiotic and ARGs in the organic solid waste are obviously better than that of the conventional thermophilic composting, the equipment investment is small, the operation is simple, so that it is very suitable for large-scale industrial operation.

The present disclosure not only realizes the removal effect of antibiotic residues and resistance gene pollution in organic solid waste, but also use the organic solid waste highly decomposed at the end of fermentation to produce organic fertilizer.

### BRIEF DESCRIPTION OF THE DRAWING

FIG. 1 shows a flow diagram of a method for reducing antibiotics and antibiotic resistance genes in organic solid waste according to one embodiment of the present disclosure.

### DETAILED DESCRIPTION OF THE EMBODIMENTS

The present disclosure is further described below with reference to the embodiments, but is not limited thereto.

In the following embodiments, the sludge used in Urban sewage treatment plant has a moisture content of about 80%. The embodiments and the control treatment test were both performed in Zhengzhou Wangxinzhuang Wastewater Treatment and Compost Plant. Three main antibiotics, tetracycline, sulfadiazine, and oxytetracycline, were tested, and nine major ARGs resistance genes, tetA, tetG, strA, strB, aacA4, aadE, ermT, mefA and ereA, were also tested at the same time.

### Example 1

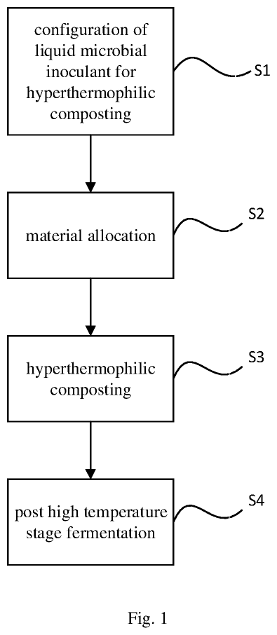

Referring to FIG. 1, the present disclosure provides a method for reducing antibiotics and antibiotic resistance genes in organic solid waste. The method includes the following steps:

S1) configuration of liquid microbial inoculant for hyperthermophilic composting: Calditerricola yamamurae UTM801, Thermophilus thermophilus FAFU013, Geobacillus sp. UTM801, Bacillus methyltrophicus UTM401 and Bacillus sp. UTM03 were activated and cultured respectively to logarithmic growth phase, and the bacteria are mixed evenly in the proportion of 1:1:1:1:1:1 by mass for reserve use.

S2) material allocation: the sludge (moisture content 80%) and the auxiliary material rice husk (moisture content 15%) are mixed according to the volume ratio (1:4). At this time, the moisture content of the mixed material is about 55%. 0.05% of the hyperthermophilic composting inoculant is added into the mixed material, and the agglomerates with the diameter greater than 10 cm are crushed, mixing evenly.

S3) hyperthermophilic composting: the mixed material is piled into the fermentation tank for high temperature aerobic composting, turning on the fan to aerate and supplying oxygen to the material, and the initial aeration rate is 25 m³·t⁻¹·h⁻¹. The temperature of the compost pile reached the highest fermentation temperature on the second day, which was about 80° C., and the aeration rate of the fan was regulated as half of the initial aeration rate, so as to keep the temperature of the compost pile fermented at 80° C. for 5 days, during which the temperature did not change much, so there was no need to turn over the compost pile.

S4) post high temperature stage fermentation: after the end of ultra-high temperature stage, the aeration rate is kept at 40% of the initial aeration rate, and the compost pile is turned once in 5-7 days. After 27 days of fermentation, the temperature dropped to room temperature, and the fermentation was finished. ARGs and antibiotic content were analyzed by regular sampling.

Two control treatment examples were set up.

Conventional high-temperature composting treatment: the sludge (moisture content 80%) and the auxiliary material rice husk (moisture content 15%) are mixed according to a certain volume ratio (1:4). At this time, the moisture content of the mixed material is about 55%. 0.05% tap water is added instead of the hyperthermophilic composting inoculant, and the agglomerates with a diameter greater than 10 cm are crushed, mixing evenly. The mixed material is piled into the fermentation tank for high-temperature aerobic fermentation, and the compost pile is turned over and aerated according to the conventional high-temperature composting process. The compost pile is turned over every 2-4 days during the former 15 days of fermentation, and the compost pile is turned over every 5-7 days after the former 15 days of fermentation. The maximum fermentation temperature of the compost pile is about 65° C., which is maintained for 5 days, and then the fermentation temperature of the compost pile starts to fall back. After 33 days of fermentation, the temperature drops to room temperature, and the fermentation is basically over.

Natural composting treatment: the sludge (moisture content 80%) and the auxiliary material rice husk (moisture content 15%) are mixed according to a certain volume ratio (1:4). At this time, the moisture content of the mixed material is about 55%. 0.05% tap water is added instead of the hyperthermophilic composting inoculant, and the agglomerates with a diameter greater than 10 cm are crushed, mixing evenly. The mixed material is placed in a cool place and stacked naturally. The maximum fermentation temperature of the compost pile can only reach about 50° C., and it will fall back after 1 day. After 33 days of fermentation, the temperature drops to room temperature, and the fermentation is basically over.

The contents of ARGs and antibiotics were analyzed by regular sampling during composting.

In Example 1 and the control grouping method, three parallel tests are set up, and the test results are shown in Table 1 and Table 2.

TABLE 1
Comparison of ARGs removal efficiency of different sludge treatment processes

| Different | ARGs removal rate (%) | | | | | | | | |
|---|---|---|---|---|---|---|---|---|---|
| treatments (° C.) | tetA | tetG | strA | strB | aacA4 | aadE | ermT | mefA | ereA |

The fourth day

| | | | | | | | | | |
|---|---|---|---|---|---|---|---|---|---|
| The method provided by | 93.97 | 97.13 | 97.91 | 97.7 | 98.24 | 96.97 | 98.66 | 98.35 | 98.96 |
| present disclosure (81° C.) | | | | | | | | | |
| Conventional thermophilic | 13.09 | 15.05 | 9.59 | −56.32 | 17.56 | 16.02 | 6.4 | −26.43 | 13.71 |
| composting (38° C.) | | | | | | | | | |
| Natural composting | 2.62 | 11.01 | 11.92 | −41.27 | 9.51 | 11.21 | 13.28 | −15.29 | 10.74 |
| treatment (37° C.) | | | | | | | | | |

The Fifteenth day

| | | | | | | | | | |
|---|---|---|---|---|---|---|---|---|---|
| The method provided by | 95.5 | 99.5 | 99.35 | 99.27 | 99.72 | 98.98 | 99.6 | 98.53 | 99.88 |
| present disclosure (62° C.) | | | | | | | | | |
| Conventional thermophilic | 26.6 | 66.82 | 64.81 | −61.52 | 62.13 | 62.33 | 56.22 | −26.78 | 52.67 |
| composting (65° C.) | | | | | | | | | |
| Natural composting | 7.3 | 12.59 | 14.26 | −12.94 | 13 | 14.17 | 14.44 | −19.22 | 13.7 |
| treatment (50° C.) | | | | | | | | | |

Twenty-seventh day

| | | | | | | | | | |
|---|---|---|---|---|---|---|---|---|---|
| The method provided by | 94.22 | 93.23 | 96.6 | 98.11 | 98.49 | 96.79 | 94.01 | 96.85 | 99.7 |
| present disclosure (27° C.) | | | | | | | | | |
| Conventional thermophilic | 36.52 | 62.97 | 71.29 | −54.68 | 64.98 | 70.86 | 72.18 | −46.08 | 68.69 |
| composting (43° C.) | | | | | | | | | |
| Natural composting | 11.91 | 14.39 | 15.88 | −15.39 | 14.8 | 12.83 | 12.39 | −29.48 | 15.64 |
| treatment (41° C.) | | | | | | | | | |

In Example 1, the hyperthermophilic composting provided by the present disclosure is adopted, the temperature of the compost pile reaches the maximum fermentation temperature of about 80° C. on the second day, and the high temperature of 80° C. is maintained for at least 5 days by regulating the aeration rate, so as to fully remove the pollution of residual antibiotic resistance genes in the sludge. It can be seen from Table 1 that the treatment method of Example 1 can quickly and efficiently remove all the detected ARGs in the sludge, and the removal rate reach more than 90% on the fourth day, and the removal effect is significantly higher than that of the other two control treatments. Although the temperature of the compost pile has dropped to normal temperature at the end of fermentation, the abundance of ARGs still hasn't rebounded, indicating that the method of the present disclosure can stably and effectively reduce the pollution of antibiotic resistance genes in sludge.

The removal efficiency of ARGs in conventional high-temperature composting was significantly lower than that in ultra-high temperature composting, although the maximum temperature was 65° C. after 15 days and maintained for 5 days. With the end of fermentation, the temperature of the compost decreased, a large number of ARGs rebounded, especially the antibiotic resistance genes of strB and mefA increased, which indicated that conventional high temperature compost could not remove ARGs quickly and stably. The highest fermentation temperature of natural composting can only reach about 50° C., and only lasts for 1 day. The removal effect of ARGs is the same as that of conventional high temperature composting, and the effect is poor.

TABLE 2
Comparison of antibiotic removal effects of different treatments

| Different | | | |
|---|---|---|---|
| treatments | tetracycline | sulfadiazine | oxytetracycline |
| The method | 99% | 98% | 99% |
| provided by present | | | |
| disclosure | | | |
| Conventional | 56% | 43% | 45% |
| thermophilic | | | |
| composting | | | |
| Natural composting | 5% | 20% | 22% |
| treatment | | | |

It can be seen from Table 2 that the treatment method by the present disclosure can quickly and efficiently remove three kinds of antibiotics from the sludge, and the removal rate is over 98%, and the removal effect is significantly higher than the other two control treatments. The results show that the method can effectively remove the antibiotic residues in the sludge.

16S rRNA gene high-throughput sequencing method was used to compare the microbial community structure of ARGs at the end of fermentation of conventional high-temperature compost and ultra-high temperature compost. The results are shown in Table 3.

TABLE 3
Comparison of the content of microorganisms carrying
ARGs in different treatments

| | | | ARGs non carrier |
|---|---|---|---|
| | ARGs carrier host | | host |
| | (potential) | | Phylum of |
| Different | Phylum of | Phylum of | Deinococcus- |
| treatments | Bacteroidetes | Proteobacteria | Thermus |
| The material before | 27% | 35% | 0.30% |
| fermentation | | | |
| The method | 0.30% | 1% | 53% |
| provided by present | | | |
| disclosure | | | |
| Conventional | | | |
| thermophilic | 17% | 20% | 0.70% |
| composting | | | |
| Natural composting | | | |
| treatment | 26% | 29% | 0.20% |

The results of Table 3 show that the addition of super thermophilic bacteria can significantly increase the proportion of non ARGs host extreme thermophilic bacteria (Deincoccus-Thermus), significantly reduce the proportion of ARGs host bacteria (Proteobacteria and Bacteroidetes), so as to enhance the removal effect of ARGs and ensure that the resistance gene abundance does not rebound at the end of fermentation. On the contrary, at the end of conventional high-temperature composting and natural composting, although the temperature rises to more than 50° C., the abundance of ARGs host of the original microbial community in the sludge is still high, which is the main reason for the poor removal effect.

In summary, the hyperthermophilic composting of the present disclosure can not only rapidly degrade antibiotics and ARGs, but also change the whole microbial community structure in the compost, kill 90% of the microorganisms carrying ARGs, reduce the risk of gene transfer of ARGs, control the spread of ARGs from the source, and ensure that ARGs will not rebound.

### Comparative Example 1

The difference from Example 1 is that the initial aeration rate is 5 m³·t⁻¹·h⁻¹. In this example, because the initial aeration rate is too small, the temperature of the compost pile rises slowly, reaching about 70° C. after 6 days, and the aeration rate of the fan is regulated to 80% of the initial aeration rate, resulting in the maximum temperature being maintained for only 4 days. The final removal rate of ARGs is below 76%.

### Comparative Example 2

The difference from Example 1 is that the initial aeration rate is 30 m³·t⁻¹·h⁻¹. In this example, after the temperature of the compost pile reached 80° C. on the third day, since the aeration rate does not regulate, the high temperature retention time was shortened to 3 to 4 days. After the end of the high-temperature phase, aeration was no longer provided, and the compost pile was turning over only once a week. The results showed that the entire fermentation cycle was prolonged for 5-7 days, and the removal rate of ARGs after 27 days was 81%, which was significantly lower than that of 96% in Example 1.

The above embodiment is a preferred embodiment of the present disclosure, but the embodiment of the present disclosure is not limited by the above embodiment. Any other changes, modifications, substitutions, combinations, and simplifications made without departing from the spirit and principle of the present disclosure, which should be regarded as equivalent replacement methods, are included in the scope of the present disclosure.

What is claimed is:

1. A method reducing antibiotics and antibiotic resistance genes in organic solid waste, comprising administering to the organic solid waste an effective amount of a hyperthermophilic composting; wherein an effective amount of hyperthermophilic composting microbial inoculant comprising aerobic microorganisms, which are able to tolerate at least 80° C., is added into the organic solid waste for hyperthermophilic composting, and a temperature of the material is controlled to be not less than 80° C. for fermentation for at least 5 to 7 days.

2. The method according to claim 1, wherein the aerobic microorganisms which are able to tolerate at least 80° C. comprises at least one of Thermophilic bacteria Calditerricola yamamurae, Thermus thermophilus, Geobacillus sp., Bacillus methylotrophicus, and Bacillus sp.

3. The method according to claim 2, wherein the hyperthermophilic composting microbial inoculant comprising the aerobic microorganisms that are able to tolerate at least 80° C. is one or more selected from the group consisting of Calditerricola yamamurae UTM801 deposited as CGMCC No. 6185, Thermus thermophilus FAFU013 deposited as CGMCC No. 14654, Geobacillus sp. UTM801 deposited as CGMCC No. 5641, Bacillus methylotrophicus UTM401 deposited as CGMCC No. 5927, and Bacillus sp. UTM03 deposited as CGMCC No. 5643.

4. The method according to claim 1, wherein the organic solid waste comprises sludge, livestock and poultry manure, and kitchen waste.

5. The method according to claim 1, wherein the antibiotic comprises tetracycline, sulfadiazine, and oxytetracycline; and the resistance gene comprises tetA, tetG, strA, strB, aacA4, aadE, ermT, mefA, and ereA.

6. A method for rapidly reducing antibiotics and antibiotic resistance genes in organic solid waste, comprising adding an effective amount of hyperthermophilic composting microbial inoculant composed of aerobic microorganisms that are able to tolerate at least 80° C. into a organic solid waste for hyperthermophilic composting, and controlling the temperature of the material to be not less than 80° C. for fermentation for at least 5 to 7 days.

7. The method according to claim 6, wherein when conducting the hyperthermophilic composting, regulating the aeration rate and controlling a count of compost turning; regulating the aeration rate and controlling the count of compost turning comprises:
controlling the initial aeration rate to 20 to 40 m³·t⁻¹·h⁻¹; regulating the aeration rate to 45-75% of the initial aeration rate after the material reaches the maximum temperature of 80° C. or above, keeping the temperature at 80° C. or above 80° C. for 5 to 7 days and no compost turning is performed during this process; regulating the aeration rate to 35-50% of the initial amount after the hyperthermophilic composting above 80° C. is completed, and turning compost pile once in 5-7 days; when the temperature drops to room temperature, the fermentation is ended.

8. An hyperthermophilic composting microbial inoculant, comprising at least one of Calditerricola yamamurae, Thermus thermophilus, Geobacillus sp., Bacillus methylotrophicus, and Bacillus sp.

9. The hyperthermophilic composting microbial inoculant according to claim 8, wherein the hyperthermophilic composting microbial inoculant is one or more selected from the group consisting of Calditerricola yamamurae UTM801 deposited as CGMCC No. 6185, Thermus thermophilus FAFU013 deposited as CGMCC No. 14654, Geobacillus sp. UTM801 deposited as CGMCC No. 5641, Bacillus methylotrophicus UTM401 deposited as CGMCC No. 5927, and Bacillus sp. UTM03 deposited as CGMCC No. 5643.

10. The hyperthermophilic composting microbial inoculant according to claim 8, wherein the hyperthermophilic composting microbial inoculant is utilized in reducing antibiotics and antibiotic resistance genes in organic solid waste.
